(19) United States
(12) Patent Application Publication
(10) Pub. No.: US 2022/0395338 A1
(43) Pub. Date: Dec. 15, 2022

(54) DETECTING A TRIGGER IN A SURGICAL ROBOTIC SYSTEM

(71) Applicant: CMR SURGICAL LIMITED, Cambridge, Cambridgeshire (GB)

(72) Inventors: Paul Christopher Roberts, Cambridge, Cambridgeshire (GB); Simon Schofield, Cambridge, Cambridgeshire (GB); Hamish Henderson, Cambridge, Cambridgeshire (GB)

(21) Appl. No.: 17/779,438

(22) PCT Filed: Jun. 22, 2020
(86) PCT No.: PCT/GB2020/051502

§ 371 Date: May 24, 2022

(30) Foreign Application Priority Data
Nov. 29, 2019 (GB) 1917437.4

(51) Int. Cl. A61B 34/30

(52) U.S. Cl. CPC A61B 34/30; A61B 2090/0804

(57) ABSTRACT

A surgical robotic system for identifying the triggering of a condition in the system, the system comprising: a first robot arm; a controller; and a first wiring arrangement configured to provide an electrical connection between the first robot arm and the controller, the first wiring arrangement comprising: a first electrical coupling comprising circuitry configured to generate a selective electrical disconnect; a second electrical coupling; a first sensor configured to measure a first electrical output from the first electrical coupling; and a second sensor configured to measure a second electrical output from the second electrical coupling; wherein the controller is configured to detect the triggering of the condition by comparing the first electrical output and the second electrical output.

### FIELD OF THE INVENTION

This invention relates to controlling robot arms, and in particular to detecting the activation of a protective stop function in a robot arm.

### BACKGROUND OF THE INVENTION

Surgical robotic systems are currently being developed for performing operations on human patients. These systems typically comprise one or more surgical robots with robot arms that are remotely controlled by a surgeon. The surgeon controls the individual surgical robots from behind a console and uses these robots to manipulate the body of the patient. The use of surgical robotic systems provides a number of advantages to patients including shorter hospitalisation, reduced pain and discomfort, faster recovery times and minimal scarring. However, in order for these robots to be a viable alternative to and replacement for human surgeons they must be resistant to errors and must be able to execute a high level of system control whilst they are in operation.

Surgical robots that are designed to be used within a surgical robotic system may comprise a protective stop mechanism, which is introduced to ensure that a robot can be safely stopped if a specific triggering condition is encountered. Triggering conditions may be external to the robot, such as the anticipated collision of the robot with another robot in the robotic system, or alternatively may be internal to the robot, such as a cable fault. When the protective stop is activated, the motion of the robot is stopped but current continues to flow through its motors. If the protective stop is activated during surgery, this function provides the surgeon with a time window in which to address the triggering condition, if this is possible, without incurring damage to the patient that could be caused by an emergency stop. In order to apply a protective stop the system, there must be an apparatus for detecting that this function has been activated in one or more of the surgical robots.

### SUMMARY OF THE INVENTION

According to a first aspect, there is provided a surgical robotic system for identifying the triggering of a condition in the system, the system comprising: a first robot arm; a controller; and a first wiring arrangement configured to provide an electrical connection between the first robot arm and the controller, the first wiring arrangement comprising: a first electrical coupling comprising circuitry configured to generate a selective electrical disconnect; a second electrical coupling; a first sensor configured to measure a first electrical output from the first electrical coupling; and a second sensor configured to measure a second electrical output from the second electrical coupling; wherein the controller is configured to detect the triggering of the condition by comparing the first electrical output and the second electrical output.

The first electrical coupling may further comprise a first cable pairing and a second cable pairing.

The second electrical coupling may further comprise a third cable pairing and a fourth cable pairing.

The cable pairings may be ethernet pairings.

The circuitry of the first electrical coupling may comprise a switch that is configured such that, when it is not activated, it is in a closed configuration.

The circuitry of the first electrical coupling may further comprise a limiter that is configured to vary the current passing through the circuit.

The second electrical coupling may further comprise a fixed resistor.

The controller may be configured to detect the triggering of the condition in response to the issuance of a protective stop function by the surgical robot.

The controller may be comprised within a surgeon's console.

The system may further comprise a plurality of robot arms.

The first wiring arrangement may be connected at a first end to a second wiring arrangement and at a second end to a third wiring arrangement, wherein; the second wiring arrangement is configured to provide an electrical connection between a second robot arm and the controller; and the third wiring arrangement is configured to provide an electrical connection between a third robot arm and the controller.

The first wiring arrangement may be electrically isolated from both the second wiring arrangement and the third wiring arrangement.

The first wiring arrangement may be coupled at the first and second ends to the second wiring arrangement and the third wiring arrangement, respectively, by transformers.

The electrical couplings of the first wiring arrangement, the second wiring arrangement and the third wiring arrangement may be arranged in parallel.

The first sensor may be configured to detect the selective electrical disconnect in the first electrical coupling.

The second sensor may be configured to provide an indication that the first robot arm is electrically connected to the surgical robot.

The first current may be applied to the first electrical coupling and a second current may be applied to the second electrical coupling, the first and second electrical currents being equal in value.

The first and second electrical outputs may be voltage.

The first and second electrical outputs may be current.

According to a second aspect, there is provided a method for detecting the triggering of a condition in a surgical robotic system, the method comprising; applying a first current to a first electrical coupling of a first wiring arrangement, the first wiring arrangement being configured to provide an electrical connection between the first robot arm and a controller, the first electrical coupling comprising circuitry configured to generate a selective electrical disconnect; applying a second current to a second electrical coupling of the first wiring arrangement measuring a first electrical output from the first electrical coupling; measuring a second electrical output from the second electrical coupling; and detecting the triggering of the condition by comparing the first electrical output and the second electrical output.

### DETAILED DESCRIPTION

The present invention will now be described by way of example with reference to the accompanying drawings. In the drawings:

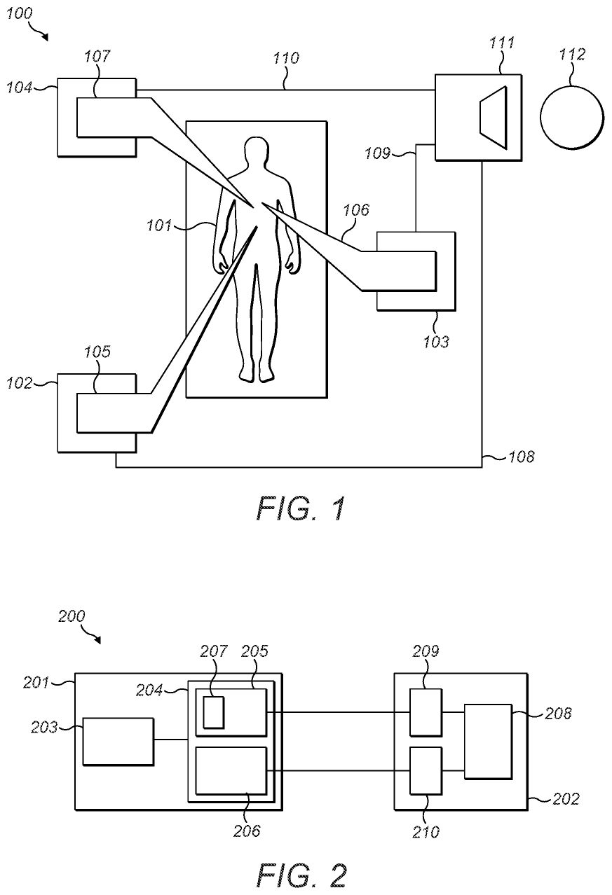

FIG. 1 illustrates a surgical robotic system;

FIG. 2 illustrates a system for identifying the activation of a protective stop in a surgical robot;

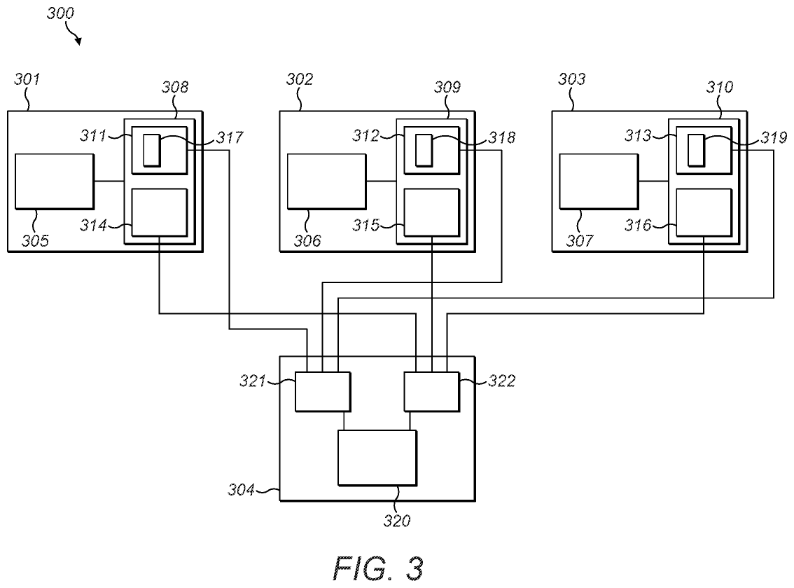

FIG. 3 illustrates a system for identifying the activation of a protective stop in a surgical robotic system comprising multiple surgical robots;

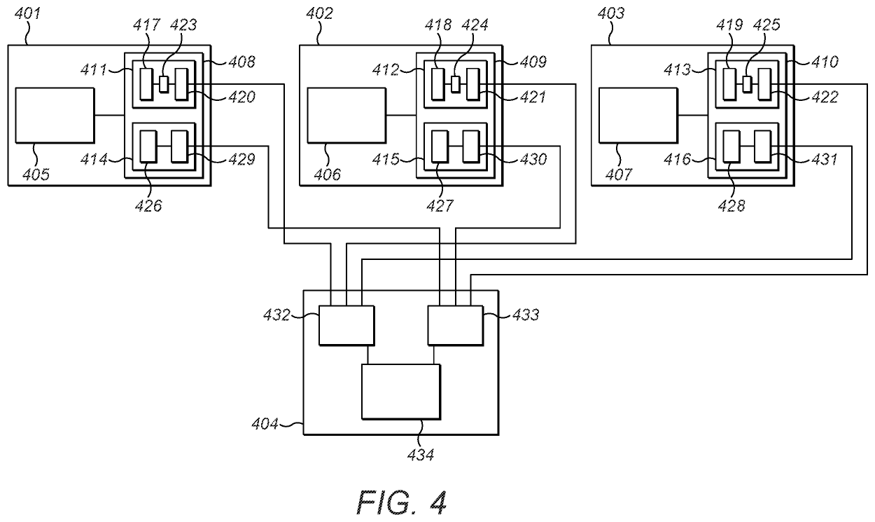

FIG. 4 illustrates a more detailed example of the surgical robotic system illustrated in FIG. 3;

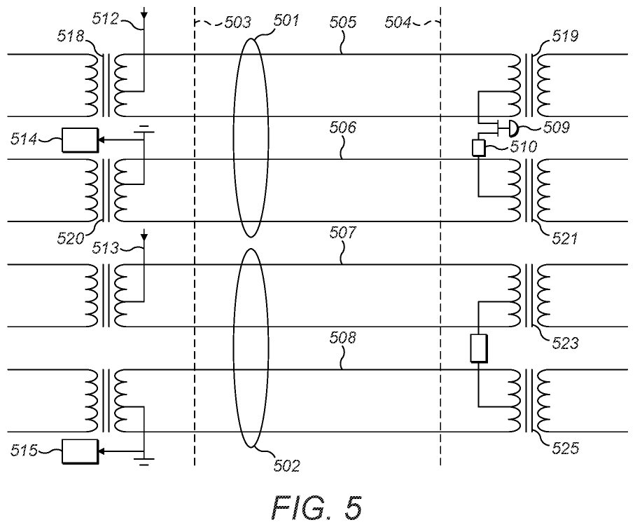

FIG. 5 illustrates a circuit diagram of an example of the surgical robotic system illustrated in FIG. 4;

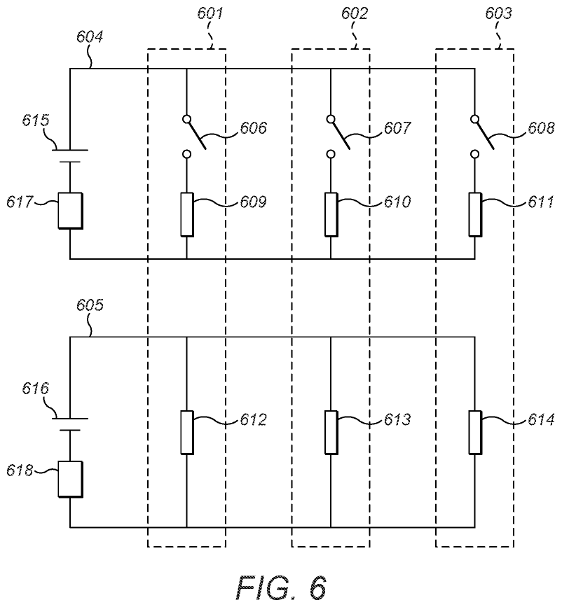

FIG. 6 illustrates a circuit diagram of an alternative example of the surgical robotic system illustrated in FIG. 4;

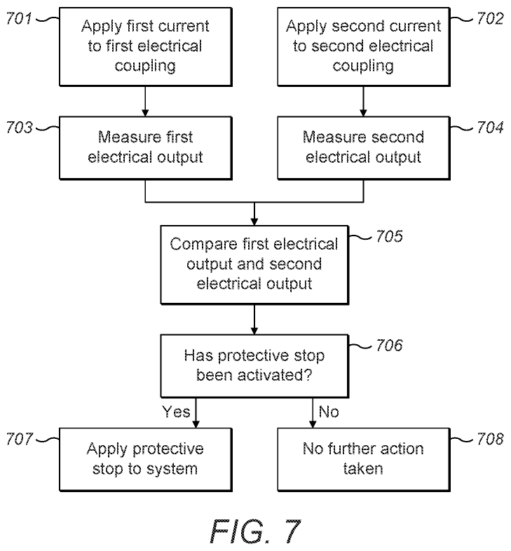

FIG. 7 is a flow chart illustrating a method for identifying the triggering of a protective stop function.

The following disclosure relates to a surgical robotic system 100 of the type illustrated in FIG. 1. The robotic system 100 is for performing surgery on a patient 101, and comprises a plurality of surgical robots 102, 103, 104. Although in FIG. 1 the robotic system is illustrated as comprising three surgical robots, it will be appreciated that the system may alternatively comprise any number of surgical robots. Each surgical robot 102, 103, 104 comprises a robot arm 105, 106, 107 which is for performing surgery on the patient 101 and sensing circuitry for detecting internal or external faults relative to the arm. The robots 102, 103, 104 are also connected, via an electrical connection such as a cable 108, 109, 110, to a surgeon's console 111. The surgeon's console 111 is operated by a surgeon 112 and is used to control the movements of the robot arms 105, 106, 107 of the surgical robots 102, 103, 104.

The surgical robots 102, 103, 104 each comprise a protective stop, which is configured to stop movement of the respective arm 105, 106, 107 of each robot relative to the patient 101 on detection of a triggering condition. The protective stop function differs from an emergency stop function in that, whilst it stops the motion of the robot, there is still current passing through its circuitry. This function is important for use in robots with surgical applications as the robots may be inside the body of a patient when the protective stop is triggered. Thus, it is preferable that the arm of the surgical robot is held in place and is not merely shut down, which might cause unwanted damage to the patient. A triggering condition may be activated automatically by the detection of a triggering condition by the sensing circuitry that is located on each of the arms. Alternatively, the protective stop may be activated by the surgeon or operating room staff on observation of an anticipated triggering condition. Each surgical robot 102, 103, 104 of the surgical robotic system should comprise its own protective stop, so that the safety of each robot can be assessed and accounted for independently. The protective stop for each robot must be fail-safe, so that if any component of the robot fails, it can be safely stopped.

If the protective stop of one of the surgical robots 102, 103, 104 in the surgical robotic system has failed, it is also important to stop the remaining robots of the system in addition to stopping the robot that has failed to ensure that the error that has been detected for one robot will not impact the operation for the others. In addition to this, applying a protective stop to the system in its entirety will allow the surgeon time to address the trigger that has activated the protective stop function without having to continue to operate the system.

FIG. 2 illustrates an example system 200 for detecting the activation of a protective stop in a surgical robot. This example system comprises one surgical robot 201 that is electrically connected to a surgeon's console 202. In this example, some components of the system 200 are comprised with the surgical robot 201 and other components are comprised within the surgeon's console 202. The surgical robot 201 may correspond to any of the robots 102, 103, 104, and the surgeon's console 202 may correspond to console 111, as illustrated in FIG. 1. The surgical robot 201 of the system comprises a robot arm 203 and a first wiring arrangement 204. The first wiring arrangement 204 is electrically connected to both the robot arm 203 and the surgeon's console 202. The wiring arrangement 204 further comprises a first electrical coupling 205 and a second electrical coupling 206. The first electrical coupling 205 comprises circuitry 207 that is configured to generate a selective electrical disconnect. Whilst the wiring arrangement 204 is illustrated in FIG. 2 as being comprised within the surgical robot 201, it will be appreciated that one or more of the components of this wiring arrangement may alternatively be located externally to the surgical robot.

The surgical robot 201 further comprises a protective stop, which is configured to stop movement of the arm 202 of the robot relative to a patient on which the robot is operating on detection of a triggering condition. The protective stop may be comprised within the first wiring arrangement 204 or may be external from and electrically connected to this wiring arrangement.

The surgeon's console 202 comprises a controller 208 which is electrically connected to both the first electrical coupling 205 and the second electrical coupling 206. The surgeon's console 202 further comprises two sensors; a first sensor 209 and a second sensor 210. The first sensor 209 is electrically connected to the first electrical coupling 205 and is configured to measure a first electrical output from this coupling. The second sensor 210 is electrically connected to the second electrical coupling 206 and is configured to measure a second electrical output from this coupling. The controller 208 is electrically connected to both the first sensor 209 and the second sensor 210, and is configured to detect the triggering of a condition by comparing the first electrical output that is measured by the first sensor 209 and the second electrical output that is measured by the second sensor 210.

FIG. 3 illustrates an alternative example in which the surgical robotic system 300 comprises a plurality of surgical robots 301, 302, 303. Whilst FIG. 3 illustrates three surgical robots that are comprised within the surgical robotic system 300, it will be appreciated that any alternative number of surgical robots may be incorporated within the system. The surgical robots 301, 302, 303 may correspond to the surgical robots 102, 103, 104 illustrated in FIG. 1. Each surgical robot 301, 302, 303 of the surgical robotic system is electrically connected to a surgeon's console 304, which may correspond to console 111 illustrated in FIG. 1. In the arrangement illustrated in FIG. 3 each surgical robot 301, 302, 303 is directly connected to the surgeon's console. In an alternative arrangement of the surgical robotic system, the surgical robots may be connected in a series, or “daisy chain” arrangement. In this alternative arrangement, only the first surgical robot in the series is directly connected to the surgeon's console. In a further alternative, the surgical robots may be connected in a hybrid arrangement, comprising any combination of the two previously described arrangements. Each surgical robot 301, 302, 303 comprises a corresponding robot arm 305, 306, 307 which is located on that robot 301, 302, 303.

The first surgical robot 301 comprises a first wiring arrangement 308 that is electrically connected to both the first robot arm 305 and the surgeon's console 304. The second robot arm comprises a second wiring arrangement 309 that is electrically connected to both the second robot arm 306 and the surgeon's console 304. The third surgical robot 303 comprises a third wiring arrangement 310 that is electrically connected to both the third robot arm 307 and the surgeon's console 304. Due to the electrical connections between the first wiring arrangement, the second wiring arrangement, the third wiring arrangement and the console 304, the first wiring arrangement 308 is connected to both the second wiring arrangement 309 and the third wiring arrangement 310. Whilst the wiring arrangements 308, 309, 310 are illustrated in FIG. 3 as being comprised within corresponding surgical robots 301, 302, 303, it will be appreciated that one or more of the components of these wiring arrangements may alternatively be located externally to the surgical robots.

The first wiring arrangement 308 further comprises a first electrical coupling 311 and a second electrical coupling 314. The first electrical coupling 311 comprises circuitry 317 that is configured to generate a selective electrical disconnect. The second wiring arrangement 309 further comprises a third electrical coupling 312 and a fourth electrical coupling 315. The third electrical coupling 312 comprises circuitry 318 that is configured to generate a selective electrical disconnect. The third wiring arrangement 310 further comprises a fifth electrical coupling 313 and a sixth electrical coupling 316. The fifth electrical coupling 313 comprises circuitry 319 that is configured to generate a selective electrical disconnect.

Each surgical robot 301, 302, 303 further comprises a protective stop, which is configured to stop movement of the arms 305, 306, 307 of the robots relative to a patient on which the robot is operating on detection of a triggering condition. The protective stop may be comprised within the first wiring arrangements 308, 309, 310 of the respective surgical robots or may be external and electrically connected to these wiring arrangements.

In this example, the surgeon's console 304 comprises a controller 320 which is electrically connected to the electrical couplings 311-316. The surgeon's console 304 further comprises two sensors: a first sensor 321 and a second sensor 322. The first sensor 321 is electrically connected to the first, third and fifth electrical couplings 311, 312, 313 and is configured to measure one or more first electrical outputs from these couplings. The second sensor 322 is electrically connected to the second, fourth and sixth electrical couplings 314, 315 and 316, and is configured to measure one or more second electrical outputs from these couplings. The controller 320 is electrically connected to both the first sensor 321 and the second sensor 322. The controller 320 is configured to detect the activation of a protective stop on one or more of the surgical robots 301, 302, 303. The controller 320 does this by comparing the one or more first electrical outputs measured by the first sensor 321 with the one or more second electrical outputs measured by the second sensor 322.

The first sensor 321 may measure one electrical output that is an aggregated value of the electrical output obtained from the first, third and fifth electrical couplings. Alternatively, the sensor 321 may measure three electrical outputs: one value for each of the first, third and fifth electrical couplings. The second sensor 322 may measure one electrical output that is an aggregated value of the electrical output obtained from the second, fourth and sixth electrical couplings. Alternatively, the sensor 322 may measure three electrical outputs: one value for each of the second, fourth and sixth electrical couplings. Although in FIG. 3 the system is illustrated as comprising two sensors, it may alternatively comprise six sensors; one for measuring the electrical output from each of the electrical couplings. Any alternative number of sensors may be used to implement the measurement of electrical outputs from the surgical robots 301, 302, 303.

FIG. 4 provides a more detailed example of the surgical robotic system illustrated in FIG. 3. The system in FIG. 4 comprises three surgical robots 401, 302, 403 that may be comparable to surgical robots 301, 302, 303 as illustrated in FIG. 3. Correspondingly, the first, second and third robot arms 405, 406, 407 may be comparable to the first, second and third robot arms 305, 306, 307 of FIG. 3. The first, second and third wiring arrangements 408, 409, 410 may be comparable to the first, second and third wiring arrangements 308, 309, 310 of FIG. 3. As with the system illustrated in FIG. 3, the first wiring arrangement 408 comprises a first electrical coupling 411 and a second electrical coupling 414. The second wiring arrangement 409 comprises a third electrical coupling 412 and a fourth electrical coupling 415. The third wiring arrangement 410 comprises a fifth electrical coupling 413 and a sixth electrical coupling 416. Whilst the wiring arrangements 408, 409, 410 are illustrated in FIG. 4 as being comprised within corresponding surgical robots 401, 402, 403, it will be appreciated that one or more of the components of these wiring arrangements may alternatively be located externally to the surgical robots.

As with FIG. 3, each surgical robot 401, 402, 403 further comprises a protective stop, which is configured to stop movement of the arms 405, 406, 407 of the robots relative to a patient on which the robot is operating on detection of a triggering condition. The protective stop may be comprised within the first wiring arrangements 408, 409, 410 of the respective surgical robots or may be external from and electrically connected to these wiring arrangements.

In FIG. 4, the first electrical coupling 411 of the first wiring arrangement 408 further comprises a first cable pairing 417 and a second cable pairing 420. The first and second cable pairings are electrically connected by circuitry 423 that is configured to generate a selective electrical disconnect. The second electrical coupling 414 of the first wiring arrangement 408 further comprises a third cable pairing 426 and a fourth cable pairing 429 and does not comprise any circuitry that is configured to generate a selective electrical disconnect. Correspondingly, the third electrical coupling 412 of the second wiring arrangement 409 further comprises a fifth cable pairing 418 and a sixth cable pairing 421. The fifth and sixth cable pairings are electrically connected by circuitry 424 that is configured to generate a selective electrical disconnect. The fourth electrical coupling 415 of the second wiring arrangement 409 further comprises a seventh cable pairing 427 and an eighth cable pairing 430 and does not comprise any circuitry that is configured to generate a selective electrical disconnect. Furthermore, the fifth electrical coupling 413 of the third wiring arrangement 410 further comprises a ninth cable pairing 419 and a tenth cable pairing 422. The ninth and tenth cable pairings are electrically connected by circuitry 425 that is configured to generate a selective electrical disconnect. The sixth electrical coupling 416 of the third wiring arrangement 410 further comprises an eleventh cable pairing 428 and a twelfth cable pairing 431 and does not comprise any circuitry that is configured to generate a selective electrical disconnect.

The surgeon's console 404 comprises a first sensor 432 and a second sensor 433. The first sensor 432 is electrically connected to the first, third and fifth electrical couplings 411, 412 and 413 and is configured to measure a first electrical output from these couplings. The second sensor 433 is electrically connected to the second, fourth and sixth electrical couplings 414, 415 and 416, and is configured to measure a second electrical output from these couplings. The surgeon's console 404 further comprises a controller 434 that is electrically connected to both the first sensor 432 and the second sensor 433. The controller 434 is configured to detect the activation of a protective stop function in one or more of the surgical robots 401, 402, 403. The controller 434 does this by comparing the first electrical output that is measured by the first sensor 432 with the second electrical output that is measured by the second sensor 433. Although in FIG. 4 the system is illustrated as comprising two sensors, it may alternatively comprise six sensors; one for measuring the electrical output from each of the electrical couplings.

As with FIG. 3, the sensors 432 and 433 may measure one electrical output that is an aggregated value of the electrical output obtained from the respective electrical couplings to which they are electrically connected. Alternatively, sensors 432 and 433 may measure distinct electrical outputs for each electrical coupling. Although in FIG. 4 the system is illustrated as comprising two sensors, it may alternatively comprise six sensors; one for measuring the electrical output from each of the electrical couplings. Any alternative number of sensors may be used to implement the measurement of electrical outputs from the surgical robots 401, 402, 403.

FIG. 5 illustrates a circuit diagram of an example of the surgical robotic system illustrated in FIG. 2. FIG. 5 illustrates the first wiring arrangement 408 of FIG. 4 comprising the first electrical coupling 501 and the second electrical coupling 502. The first and second electrical couplings 501, 502 can correspond to the first and second couplings 411, 414 of the first wiring arrangement in FIG. 4. A first end of the first and second electrical couplings is located at the surgeon's console 503, which may be comparable to the console 111 as illustrated in FIG. 1. The first and second electrical couplings are therefore electrically powered by the surgeon's console 503. The couplings may be powered by the controller of the console. A second end of the first and second electrical couplings is located at the surgical robot 504, which may be comparable to any of the surgical robots 102, 103, 104 as illustrated in FIG. 1. The first electrical coupling 501 further comprises a first cable pairing 505 and a second cable pairing 506. The second electrical coupling 502 further comprises a third cable pairing 507 and a fourth cable pairing 508.

The first and second cable pairings 505, 506 are electrically connected by means of circuitry that is configured to generate a selective electrical disconnect. That is, the circuitry may be configured to selectively disconnect and reconnect the first and second cable pairings 505, 506 in dependence on its arrangement. In FIG. 5, this circuitry is illustrated as a switch 509. The switch is provided to implement the protective stop function of the surgical robot 504. The switch is located between the first and second cable pairings 505, 506 in order to generate the selective electrical disconnect between these cable two pairings. The switch may be a push switch and may be manually activated by a surgeon. Alternatively, the switch may be automatically activated by circuitry in the surgical robot 504. The switch may be any alternative type of switch that is capable of generating a break between the first and second cable pairings 505, 506. The switch may be a normally closed switch. The use of a normally closed switch enables current to be supplied between the first and second cable pairings when the surgical robot is in use and before the protective stop is activated. In an alternative arrangement, the switch 509 may be coupled to the robot arm so that the protective stop function can be activated by the operating room staff. The electrical connection between the first and second cable pairings 505, 506 further comprises a current limiter. In FIG. 5, this current limiter is illustrated as a resistor 510. The resistor 510 may be a variable resistor. The variable resistor may be used to control the current passing through the circuit, and to enable the circuitry of the first electrical coupling 501 to adapt to changes in current that pass through the circuit as a result of the switch 509 being opened.

In FIG. 5, the resistor 510 is illustrated as being connected in series with the switch 509. In an alternative arrangement, the resistor 510 may be connected in parallel with the switch 509. In this alternative arrangement, current is still able to flow around the circuit when the switch 509 is closed. This is advantageous as it enables the activation of the protective stop function to be distinguished from a cable break. That is, the activation of protective stop can be identified by a predetermined drop in current (such as by 4 mA, for example), as opposed to a complete break in current flow (0V).

The third and fourth cable pairings 507, 508 are electrically connected by means of a common circuitry; however, this circuitry does not comprise a component that is configured to generate a selective electrical disconnect. Thus, the third and fourth cable pairings are always electrically connected to each other such that current can pass from the third pairing 507 to the fourth pairing 508. The circuitry that is configured to electrically connect the third and fourth cable pairings comprises a current limiter. In FIG. 5 this current limiter is illustrated as a resistor 511. This resistor may be a fixed resistor. A variable resistor is not required between the third and fourth cable pairings as the connection between these pairings does not comprise circuitry for generating a selective electrical disconnect, and so current control is not required.

The robot arm of the surgical robotic system may be powered up by the surgeon's console, or alternatively may be powered by a second arm in the surgical robotic system. The second of these alternatives will occur when the surgical robotic system is arranged such that the robot arms are connected in series, or in a “daisy chain” arrangement. When the robot arm is powered up, a first current I₁ is supplied to the first electrical coupling 501 at the first end of the first cable pairing 505. This first current may also be referred to as a centre tap and is illustrated by reference numeral 512. When the switch 509 is closed, current can flow from the first cable pairing 505 to the second cable pairing 506 of the first electrical coupling 501. A second electrical current I₂ is also supplied to the second electrical coupling 502 at the first end of the third cable pairing 507. This second current is illustrated by reference numeral 513. The second current can flow from the third cable pairing 507 to the fourth cable pairing 508 of the second electrical coupling 502.

The surgeon's console comprises a first sensor 514 and a second sensor 515. The first and second sensors 514, 515 may correspond to first and second sensors 432, 433 in FIG. 4. The first sensor 514 is electrically connected to the second cable pairing 506 of the first electrical coupling 501 at its first end and is configured to measure a first electrical output from the first electrical coupling 501. In the example illustrated in FIG. 5, this first electrical output is a voltage V₁. As the first sensor 514 is electrically connected to the second cable pairing 506, and the second cable pairing 506 is located at the output of the switch 509, the voltage V₁ that is measured by the sensor 514 changes in dependence on whether the switch is open or closed. More specifically, if the switch is closed the voltage V₁ has a first non-zero value and if it is open it has a second value. In the arrangement illustrated in FIG. 5, this second value will be zero. In an alternative arrangement where the resistor 510 is connected in parallel with the switch 509, the second value will be a non-zero value that is lower than the first non-zero value. The first sensor 514 is therefore able to detect a selective electrical disconnect in the first electrical coupling 501 that is activated by the opening of the switch 509. The second sensor 515 is electrically connected to the fourth cable pairing 508 of the second electrical coupling 502 at its first end and is configured to measure a second electrical output from the second electrical coupling 502. In the example illustrated in FIG. 5, this second electrical output is a voltage V₂, which is illustrated by reference numeral 515.

The wiring arrangement illustrated in FIG. 5 is associated with a first surgical robot. The wiring arrangement may be electrically coupled at its first end to the second wiring arrangement of a second surgical robot. The wiring arrangement may also or alternatively be coupled at its second end to the wiring arrangement of a third surgical robot. In this example, the second wiring arrangement is configured to provide an electrical connection between a second robot arm and the controller, and the third wiring arrangement is configured to provide an electrical connection between the third robot arm and the controller. In FIG. 5, the wiring arrangement comprises circuitry configured to electrically isolate the first, second, third and fourth cable pairings from the cable pairings of the second and/or third wiring arrangements of the second and third surgical robots. More specifically, each cable pairing comprises a first transformer 518, 520, 522, 524 at its first end and a second cable pairing 519, 521, 523, 525 at its second end. Isolation is achieved from this configuration as electricity cannot pass from one side of the transformer to the other. This isolation prevents high currents from being passed through the neighbouring wiring arrangements to the patient or to safety critical components of the robot arm. This prevents damage to either the patient or to the surgical robotic system.

The first and second sensors 514, 515 are connected to a controller (not shown). The controller is configured to compare the electrical outputs that are measured by these sensors. The second voltage V₂ from the second electrical coupling 502 that is measured by the second sensor 515 has a non-zero value when the surgical robot is being powered by the surgeon's console. As the second electrical coupling 502 does not comprise circuitry configured to generate a selective electrical disconnect, the second voltage V₂ should remain constant as long as the surgical robot 502 is powered by the console 503. The first voltage V₁ is non-zero and is the same value as V₂ when the surgical robot is powered by the system unless the protective stop is activated. In the arrangement of the surgical robotic system illustrated in FIG. 5, when the protective stop is activated, and the normally closed switch is opened, current can no longer pass to the second cable pairing 506 of the first electrical coupling. In an alternative arrangement where the resistor is connected in parallel with the normally closed switch, input current passes through the resistor as opposed to the switch. This results in a reduction in output current.

Using the method described above, the second sensor 515 can be used to provide an indication that the robot arm 504 is being powered by the console 503. If the value of the second voltage V₂ is non-zero, then the robot arm is being powered. If the second voltage V₂ is zero, then the robot arm is not being powered. The first sensor 514 is used to detect whether the protective stop of the surgical robot has been activated. More specifically, protective stop is activated when the robot arm is being powered by the console but the switch 509 has been activated. Thus, protective stop has occurred in the specific instance when V₁ is zero and V₂ is non-zero. If the controller determines that there is a non-zero difference in voltage between V₁ and V₂, then the protective stop has been activated. Alternatively, the controller may simply measure the voltages V₁ and V₂ and may individually compare these voltages to predetermined expected values that are stored at the surgeon's console to determine whether the protective stop has been activated.

The cable pairings 505-508 may be ethernet pairings, such that the combination of the cable pairings forms an ethernet cable. Ethernet cables advantageously provide bandwidth and reliability improvements over alternative cable types. These cables are also easy to customise, which provides design improvements over other commercial alternatives.

FIG. 6 illustrates a circuit diagram of an alternative example of the system illustrated in FIG. 4 to that which is illustrated in FIG. 5. In FIG. 6 the first and second wiring arrangements of each surgical robot of the system are arranged in two separate electrical circuits, such that the first, third and fifth electrical couplings are arranged in parallel and the second, fourth and sixth electrical couplings are arranged in parallel. The first, second and third wiring arrangements of the first, second and third surgical robots are illustrated as reference numerals 601, 602 and 603 respectively. The first, second and third surgical robots 601, 602, 603 may correspond to the first, second and third robots 401, 402, 403 of FIG. 4. The first, third and fifth electrical couplings of the first, second and third wiring arrangements are arranged in parallel in a first electrical circuit 604. The second, fourth and sixth electrical couplings of the first, second and third wiring arrangements are arranged in parallel in a second electrical circuit 605.

Each electrical coupling of the first electrical circuit 604 comprises circuitry configured to generate a selective electrical disconnect 606, 607, 608 and current limiter 609, 610, 611. That is, each circuitry 606, 607, 608 may be configured to selectively disconnect and reconnect the flow of current to its respective current limiter 609, 610, 611 in dependence on its arrangement. The circuitry that is configured to generate a selective electrical disconnect may be a switch. The switch may be normally closed, so that current can pass through each electrical coupling of the circuit when its corresponding switch has not been activated. The switch is provided to implement the protective stop function of each surgical robot of corresponding wiring arrangements 601, 602, 603. The current limiter may be a resistor, and in particular may be a variable resistor. The variable resistor may be used to control the current passing through the circuit, and to enable the circuitry of the first electrical circuit 604 to adapt to changes in current that pass through the circuit as a result of one or more of the switches 606, 607, 608 being opened. The limiters of each wiring arrangement 601, 602, 603 in the first electrical circuit 604 may be substantially the same.

Each electrical coupling of the second electrical circuit 605 also comprises a current limiter 612, 613, 614. The current limiter may be a resistor, and in particular may be a fixed resistor. A fixed resistor can be used in the second electrical circuit 605 as this circuit does not comprise any circuitry configured to generate a selective electrical disconnect and so should not be subjected to sudden unexpected changes in current value. The resistors of each wiring arrangement 601, 602, 603 in the second electrical circuit 605 may be of the same or substantially the same resistance value.

The first and second electrical circuits 604 and 605 are each powered by a power source 615, 616. The power sources may be located at a surgeon's console, which may correspond to console 111 as illustrated in FIG. 1. The first electrical circuit 604 further comprises a first sensor 617 for measuring a first electrical output from the circuit 604. The second electrical circuit 605 further comprises a second sensor 618 for measuring a second electrical output from the circuit 605. The first and second sensors 617, 618 may be current sensors and are configured to measure current in the first and second electrical circuits respectively. The first and second sensors 617, 618 may also be located at the surgeon's console, with the power sources.

In use, a first voltage V₁ is applied to the first electrical circuit 604 by the power source 615 and a second voltage V₂ is applied to the second electrical circuit 605 by the power source 616. These voltages induce a first initial current I₁ in the first electrical circuit 604 and a second initial current I₂ in the second electrical circuit 605. As the first and second circuits are arranged in parallel, the value of current in each of these circuits is equal to the sum of the individual values of current that pass through the parallel lines of each electrical coupling of the circuit. As the second electrical circuit 605 does not comprise any circuitry that is configured to generate a selective electrical disconnect, and assuming that the fixed resistance of each of the resistors 612, 613, 614 is the same or substantially the same, the current I₂ that passes through the circuit 605 determines how many surgical robots are connected to the system. The first electrical circuit 604 is used to determine whether a protective stop has been activated in one or more of the surgical robots that are connected to the system. When the protective stop is activated in a wiring arrangement 601, 602, 603, the normally closed switch associated with this robot is opened. This breaks the flow of current through one of the parallel lines in the circuit 604. The current therefore does not pass through the resistor that is located on the same parallel line as the open switch, and so the overall resistance in the circuit decreases. For example, if the switch 606 of the first wiring arrangement 601 is opened, current does not pass through the corresponding resistor 609 of this wiring arrangement. Thus, only the resistors 610 and 611 of the second and third wiring arrangements 602, 603 contribute to the overall resistance of the circuit. The decrease in resistance across the circuit 604 results in an increase in current. The current I₁ is measured by the first sensor 617.

Both the first sensor 617 and the second sensor 618 are electrically connected to a controller (not shown). The controller is configured to receive measurements recorded by both of the sensors 617, 618, and to compare these measurements. As mentioned above, the current I₂ in the second electrical circuit 605 remains constant provided that all of the surgical robots have been connected to and are therefore being powered by the system. Thus, if a difference between the first current I₁ and the second current I₂ is determined by the controller, this indicates that a protective stop has been activated in one or more of the wiring arrangements. The system of FIG. 6 therefore allows for the detection of the activation of protective stop in a surgical robot in the surgical robotic system. The specific surgical robot for which the protective stop has been activated cannot be identified from this detection alone. However, if the surgical robotic system also comprises an active communication channel, this can be used in combination with the circuitry described in FIG. 6 to identify the surgical robot that has had its protective stop activated. As with the example of the system illustrated in FIG. 5, the wiring arrangements illustrated in FIG. 6 may be ethernet cables.

FIG. 7 illustrates a method for identifying the triggering of a protective stop function in a surgical robot of a surgical robotic system. At step 701 a first current I₁ is applied to the first electrical coupling of the first wiring arrangement. At step 702 a second current I₂ is applied to the second electrical coupling of the first wiring arrangements. The application of the first and second currents at steps 701 and 702, or alternatively may be applied at different times. At step 703 a first electrical output is measured from the first electrical coupling is measured by the first sensor. At step 704 the second electrical output is measured from the second electrical coupling by the second sensor. The measurement of the first and second electrical outputs in steps 703 and 704 may be measured simultaneously, or alternatively may be measured at different times. The first and second electrical outputs may be voltage, as described with reference to the example illustrated in FIG. 5. The first and second electrical outputs may alternatively be current, as described with reference to the example illustrated in FIG. 6. At step 705 the first electrical output and the second electrical output are compared by the controller. At step 706, the controller determines whether protective stop has been activated in the surgical robot. This determination is made either by comparing the electrical outputs to each other or by comparing each output to a predetermined expected value, as described above. If it is determined that protective stop has been activated in the robot, then the method progresses to step 707 in which the controller applies a protective stop to the entire system. This may be done by activating the protective stop of each of the remaining robots in the surgical robotic system individually. If it determined that protective stop has not been activated, then at step 708 no further action is taken by the controller. The method illustrated in FIG. 7 may be repeated continuously, or alternatively at regular intervals. That is, the first and second electrical outputs may be measured continuously so long as the first and second currents are being applied to the first and second electrical couplings respectively. Alternatively, the electrical outputs may be measured at regular intervals whilst the first and second currents are being applied. In this way, the electrical outputs from the electrical couplings can be continuously compared by the controller to determine whether a protective stop has been activated.

The surgical robotic system described herein enables the determination of the surgical robotic system in its entirety that a protective stop has been activated in one of the surgical robots of the system. In situations where the protective stop of a specific robot has been activated, such as where there has been a fault in the cabling of that robot, it is preferable to stop all of the surgical robots in the system and not just the one for which the protective stop has been activated. By alerting the system in its entirety to the activation of a protective stop, the system is configured in response to stop all of the robots of the system and to prevent any further action from being taken on behalf of these robots until the incident that triggered the activation of the protective stop has been resolved.

The described system avoids the need to use a communication channel or data link of the robotic system to convey information regarding the activation of the protective stop. This could be useful if there is a problem with the communication channel or data link. In some situations, it may be an error in these circuitries that has resulted in the activation of the protective stop.

Both examples described in FIGS. 5 and 6 illustrate circuitry which does not comprise any active circuit components. The system therefore is entirely passive and so is highly resistant to failure. This is useful within the field of surgical robotics, where accurate and reliable control of the system is of high importance for the safety of a patient.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A surgical robotic system configured to identify the triggering of a condition in the system, the system comprising:
a first robot arm;
a controller; and
a first wiring arrangement configured to provide an electrical connection between the first robot arm and the controller, the first wiring arrangement comprising:
a first electrical coupling comprising circuitry configured to generate a selective electrical disconnect;
a second electrical coupling;
a first sensor configured to measure a first electrical output from the first electrical coupling; and
a second sensor configured to measure a second electrical output from the second electrical coupling;
wherein the controller is configured to detect the triggering of the condition by comparing the first electrical output and the second electrical output.

2. A system as claimed in claim 1, wherein the first electrical coupling further comprises a first cable pairing and a second cable pairing.

3. A system as claimed in claim 1, wherein the second electrical coupling further comprises a third cable pairing and a fourth cable pairing.

4. A system as claimed in claim 2, wherein the cable pairings are ethernet pairings.

5. A system as claimed in claim 1, wherein the circuitry of the first electrical coupling comprises a switch that is configured such that, when it is not activated, it is in a closed configuration.

6. A system as claimed in claim 1, wherein the circuitry of the first electrical coupling further comprises a limiter that is configured to vary the current passing through the circuit.

7. A system as claimed in claim 1, wherein the second electrical coupling further comprises a fixed resistor.

8. A system as claimed in claim 1, wherein the controller is configured to detect the triggering of the condition in response to the issuance of a protective stop function by the surgical robot.

9. A system as claimed in claim 1, wherein the controller is comprised within a surgeon's console.

10. A system as claimed in claim 1, further comprising a plurality of robot arms.

11. A system as claimed in claim 10, wherein the first wiring arrangement is connected at a first end to a second wiring arrangement and at a second end to a third wiring arrangement, wherein;
the second wiring arrangement is configured to provide an electrical connection between a second robot arm and the controller; and
the third wiring arrangement is configured to provide an electrical connection between a third robot arm and the controller.

12. A system as claimed in claim 11, wherein the first wiring arrangement is electrically isolated from both the second wiring arrangement and the third wiring arrangement.

13. A system as claimed in claim 12, wherein the first wiring arrangement is coupled at the first and second ends to the second wiring arrangement and the third wiring arrangement, respectively, by transformers.

14. A system as claimed in claim 10, wherein the electrical couplings of the first wiring arrangement, the second wiring arrangement and the third wiring arrangement are arranged in parallel.

15. A system as claimed in claim 1, wherein the first sensor is configured to detect the selective electrical disconnect in the first electrical coupling.

16. A system as claimed in claim 1, wherein the second sensor is configured to provide an indication that the first robot arm is electrically connected to the surgical robot.

17. A system as claimed in claim 1, wherein a first current is applied to the first electrical coupling and a second current is applied to the second electrical coupling, the first and second electrical currents being equal in value.

18. A system as claimed in claim 1, wherein the first and second electrical outputs are voltage.

19. A system as claimed in claim 1, wherein the first and second electrical outputs are current.

20. A method for detecting the triggering of a condition in a surgical robotic system, the method comprising:
applying a first current to a first electrical coupling of a first wiring arrangement, the first wiring arrangement being configured to provide an electrical connection between the first robot arm and a controller, the first electrical coupling comprising circuitry configured to generate a selective electrical disconnect;
applying a second current to a second electrical coupling of the first wiring arrangement
measuring a first electrical output from the first electrical coupling;
measuring a second electrical output from the second electrical coupling; and
detecting the triggering of the condition by comparing the first electrical output and the second electrical output.
